(19) United States
(12) Patent Application Publication
(10) Pub. No.: US 2005/0071192 A1
(43) Pub. Date: Mar. 31, 2005

(54) Quick notation medical reference and record system and method of use

(76) Inventor: Nada Milosavljevic, Providence, RI (US)

Correspondence Address: JENNER & BLOCK, LLP, ONE IBM PLAZA, CHICAGO, IL, 60611, US

(21) Appl. No.: 10/675,128

(22) Filed: Sep. 30, 2003

(51) Int. Cl. G06F 17/60; A61B 5/00

(52) U.S. Cl. 705/2; 600/300

(57) ABSTRACT

A medical reference and record system and method for displaying known information pertaining to a medical disorder in a standardized format and integrating with it information from a patient to facilitate evaluation and treatment of the patient and improve communication of patient information from one health care practitioner to another. Presents reference information in ways useful for training of medical students, residents, and practitioners. A user can assess the relevance of presented information to a patient and customize the presented information to the patient. Reference information and fields for entry of patient data are combined in ways useful for multiple patients and for retention in a patient's medical record. The system and method may be implemented in printed or computerized formats.

### BACKGROUND OF THE INVENTION

(1) Field of Invention

The present invention relates to medical references, health care examinations and treatments, and recording of patients' medical information. It also relates to training of health care practitioners and communication of patient information between health care practitioners.

(2) Description of the Related Art

In a busy, modern health care practice, particularly in an emergency room situation, much information regarding a patient's condition, evaluation, diagnosis, and care must be gathered, analyzed, processed, and communicated to others rapidly, accurately, and efficiently. Diagnoses must be made and verified, treatment plans must be formulated and followed, and all caregivers must work together toward the common goal of helping the patient. The need for efficient yet thorough provision of health care has grown along with the rising costs of medical treatment and hospitalization.

When a patient presents to an emergency room in a hospital, for example, health care practitioners perform triage on the patient, make a preliminary diagnosis, order and perform confirmatory diagnostic testing, and initiate a treatment plan. These practitioners must each communicate the signs observed, symptoms reported, preliminary diagnosis, tests desired, test results, and treatment plan to other persons in their medical service team as well as persons in other health care services within the hospital. Once the preliminary diagnosis is made, much of the initial diagnostic work-up and treatment plan is the same as for any other patient with the same signs, symptoms, or preliminary diagnosis. Accordingly, much of what a health care practitioner must write in a patient's chart is repetitive among patients. Yet, nothing known in the prior art addresses this redundancy and the potential that errors may be made in the transcription or communication process. If information on diseases or disorders, including common signs and symptoms and tests routinely ordered to confirm those disorders, for example, was pre-written in a format readily accessible to practitioners, much valuable time could be saved. Further, a pre-written format would avoid the problem of illegible or misread handwriting. Moreover, a pre-written, readily accessible format specific to a diagnosis is likely to be more complete and accurate than a practitioner's memory under time pressures or the stress of an emergency situation. Additionally, if the practitioner could see pertinent information about signs, symptoms, and epidemiology of a medical disorder at a glance, the practitioner might avoid pursuing a path unlikely to yield favorable results.

Moreover, medical residents fresh from school and medical students in clinical rotations are often involved in the examination of patients, the ordering of tests, and the specification of treatments. Medical residents and medical students must try to recognize, remember, and apply, often for the first time, information they were presented in classes to actual patients while under intense pressure and both physical and emotional stress from attending physicians, supervisors, patients, staff, long working hours, inadequate sleep, and other factors.

Recent statistics show that an alarming number of inadvertent errors are made by health care professionals, frequently with serious consequences. These statistics support the need for devices and methods that can prevent or minimize such mistakes. Additionally, devices and methods that can teach new practitioners, such as medical residents and medical students in clinical rotations, more about patient care and pathologies are desirable and helpful. Currently available devices or methods that may help minimize errors do not teach new practitioners why a mistake might have occurred so that the practitioners understand how to avoid the same problem later. Further, due to the extensive and ever-changing scope of the field of medicine, not everything a physician or psychiatrist needs to know to care for patients is taught in school; much information must be learned during a residency period, although a resident has severely limited time for academic pursuits.

Most books or other types of collections of medical information available today contain too much information or are too bulky, time-consuming, or awkward to be useful at a patient's bedside or under other conditions of limited time or space. Available pocket-sized references cannot be customized for an individual patient without compromising their future use, nor can they be integrated into a patient's medical chart or readily used to facilitate communications between health care professionals. A concise collection of the most pertinent medical knowledge required for immediate decision making, which can be customized for an individual patient and provide work-up and treatment guidance, is a needed tool for health care practitioners. New practitioners such as medical residents would particularly benefit from a concise presentation of medical information in a standardized format that could be carried to patients' bedsides or charts to help these new practitioners apply their recently acquired knowledge to real cases and understand why patients with certain medical conditions are tested and treated in particular ways.

### BRIEF SUMMARY OF THE INVENTION

The present invention integrates practically useful or educational information about a medical disorder with fields for data specific to a patient suspected of having the medical disorder. Useful reference information may also be included. A user of the invention can record data that pertains to a patient's identity, signs, symptoms, tests, and treatment according to the format of the invention, and can enter other data of interest as well. The invention is an apparatus providing for or displaying known information pertaining to a medical disorder in a standardized format and a method of integrating this information with information specific to an individual patient in connection with diagnosis and treatment of the patient and/or education and training of health care practitioners.

The reference device may be presented or printed in various physical forms or incorporated into computer software for electronic manipulation or transfer between users or sites.

The invention may be used as an aid by a health care practitioner, professional, student, or trainee in evaluating the cause of a patient's signs or symptoms, confirming a diagnosis, or determining the course of treatment for a patient's condition. The invention also may be used as a template for recording notes on an individual patient or a specific medical disorder. The invention may be used to facilitate communication between caregivers about a patient. The invention may also serve as a concise reference compilation of pertinent information on a particular medical disorder or collections of medical disorders. The invention may further be used as a learning tool to study or review information specific to a particular disorder or various diseases. The invention thus aids triage of patients, facilitates communication between health care professionals, and helps train new health care practitioners. The invention has application to many types of health care practices and both physical and mental abnormalities or conditions.

### BRIEF DESCRIPTION OF THE DRAWINGS

FIGS. 1A and 1B are illustrations of first and second parts, respectively, of an embodiment of the invention depicting a template with information on gallstone diseases.

FIGS. 2A and 2B are illustrations of first and second parts, respectively, of another embodiment of the invention depicting a template with information on colorectal cancer.

FIGS. 3A and 3B are illustrations of first and second parts, respectively, of another embodiment of the invention depicting a template with information on peptic ulcer disease.

FIGS. 4A and 4B are illustrations of first and second parts, respectively, of another embodiment of the invention depicting a template with information on acute appendicitis.

FIGS. 5A and 5B are illustrations of first and second parts, respectively, of another embodiment of the invention depicting a template with information on major depressive disorder.

FIGS. 6A and 6B are illustrations of first and second parts, respectively, of another embodiment of the invention depicting a template for completion by a user of the invention.

FIG. 7 illustrates an embodiment of a portion of the invention depicting reference information in the form of a calendar.

FIG. 8 illustrates another embodiment of a portion of the invention depicting reference information in the form of lists of medical laboratory tests and measurements with corresponding values or ranges of results.

FIG. 9 illustrates another embodiment of a portion of the invention depicting lists of various types of reference information useful in a medical practice.

FIG. 10 illustrates another embodiment of a portion of the invention depicting other reference information and a data gathering field.

FIG. 11 illustrates another embodiment of a portion of the invention depicting another form of reference information and data gathering field.

FIG. 12 depicts another embodiment of a portion of the invention illustrating a medical testing tool.

FIG. 13 illustrates another embodiment of a portion of the invention depicting reference information in the form of rulers for measuring electrocardiographic data. This figure also illustrates an indexing method that may be used with the invention.

FIGS. 14A and 14B illustrate the invention as embodied in the form of a coated printed page which can be marked and erased.

FIGS. 15A, 15B, 15C, and 15D illustrate the invention embodied in another form.

FIG. 16 illustrates the invention embodied in computer software for a hand-held computer.

### DETAILED DESCRIPTION OF THE INVENTION

The present invention includes a medical reference source presenting concise, practically useful, academic, or interesting information on a medical condition, disease, disorder, or pathology in a standardized user-friendly template format customizable to an individual patient. The reference source can be used by a health care practitioner or trainee to rapidly and efficiently access essential and valuable medical reference information, record data pertaining to a specific patient, and communicate information about a patient to other health care professionals. The reference is comprised of concise bits of information about a particular medical disorder, organized in a standardized manner, with areas in which a user can indicate that a particular bit of information applies to a patient or can add other information.

Embodiments of the invention may include a printed form, handbook, or other type of conveniently portable device which a user can easily carry from patient to patient or place to place or otherwise readily access during a health care practice, medical resident or medical student rotation and can use to jog the user's memory about a medical disorder, or to determine, track, or communicate to others a patient's evaluation and treatment. The invention may also be used to provide concise teaching points. The form, handbook, or other reference device created according to the invention may be of a size capable of being easily transported in a pocket of a typical laboratory coat or other uniform worn by a health care practitioner. Alternatively, the template may be embodied in a physical or electronic form compatible with insertion into a patient's medical record or chart. The invention may also be embodied as software for a portable or hand-held computer or a network computer with terminals accessible in locations where the invention would be useful.

The organization of the invention is a generally broad to specific, chronological background to follow-up format, such as is useful in evaluating and treating a medical patient. This format presents bits of information in the general order of background information (including epidemiology, description, or etiology), presenting information (including signs or symptoms), diagnostic information (including differential diagnoses or appropriate diagnostic tests), and finally treatment, prognosis, or follow-up information. Reference information may also be included in the invention to increase its usefulness, whether related to the particular disorder or not, either intertwined in the general format or separate from the general format. Information included in the invention may be selected for its practical usefulness, such as might apply to an individual patient or be able to be put into practice by a health care professional, or for its educational value, such as might be helpful in the development of a health care practitioner's knowledge or skills.

Embodiments of the invention include a template for prompting and recordation of the information a health care practitioner might normally write in a patient's record or otherwise communicate to other practitioners working with the patient after making a preliminary diagnosis, including the patient's signs and symptoms and the plan for diagnostic work-up and treatment of the patient. The invention can facilitate efficient communication from a patient to a health care practitioner and between health care professionals. The invention can train health care practitioners by providing concise and useful or interesting information about a medical disorder helpful to the trainee's understanding of the development, prevalence, presentation, diagnosis, or treatment of the disorder.

In a preferred embodiment of the invention, a health care practitioner would select a standard form pertaining to a medical disorder exhibited by a patient. The practitioner would record the patient's identifying information on the form and indicate with a mark, such as a check or “x” by a term or a circle around or underline of a term which terms on the form, such as signs, symptoms, or risk factors, are applicable to the patient. The practitioner may mark through terms not applicable to the individual patient. The practitioner would also indicate with marks the diagnostic tests that should be performed on the patient and the treatments that should be initiated. The practitioner would further record the patient's vital signs, list any medications the patient is taking, and make any other notes desired. The practitioner could then pass the customized form on to the next practitioner who will be working with the patient.

General Template Format

The invention presents practical or academic information relating to a specific medical disorder and fields for entry of individual patient information in a template format pursuant to which bits of information are categorized and presented by category. Any medical or psychological condition, pathology, disease, disorder, abnormality, or variation may be the subject of an embodiment of the invention, whether of physical, mental, psychological, psychosocial, anatomic, metabolic, physiological, infectious, immunological, degenerative, neoplastic, traumatic, congenital, acquired, or other origin.

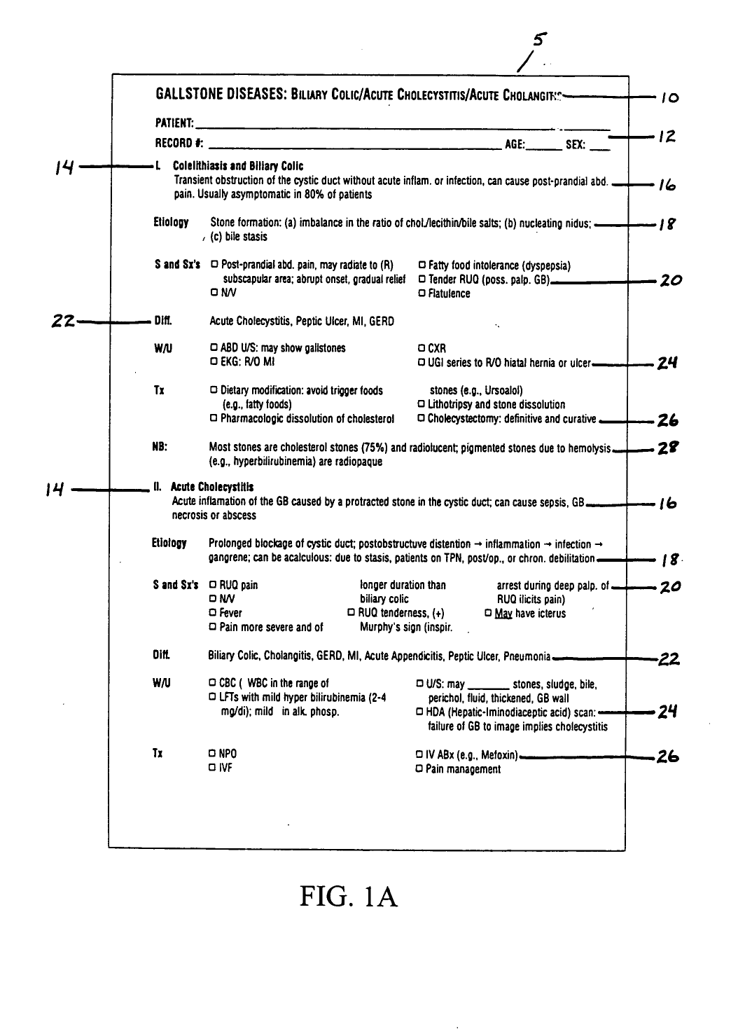

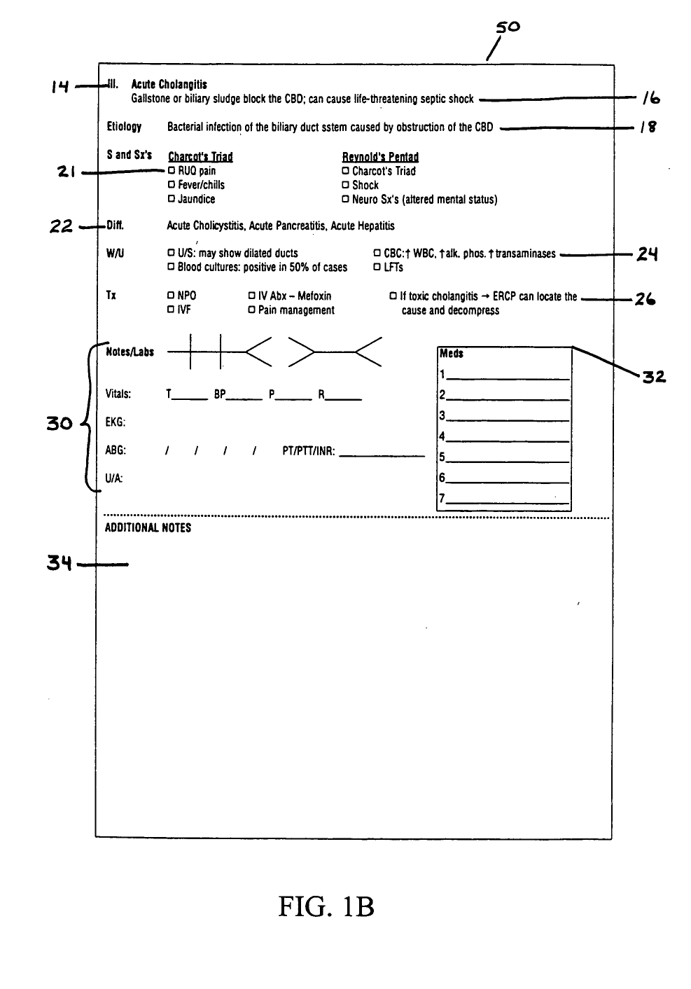

FIGS. 1A through 5B illustrate examples of presentations of information relating to a specific medical disorder. As shown by example in these figures, information categories may include the following preferred categories, which are preferably presented in this order:
Etiology
Signs and Symptoms
Differential Diagnosis
Laboratory or Other Work-Up
Treatment
   “Etiology” includes information pertaining to the cause or origin of a medical disorder. “Signs and symptoms” include visible, palpable, audible, or other abnormalities or clinical manifestations detectable by a physical examination of a patient, as well as complaints, pains, emotions, discomforts, feelings, or other sensations described by a patient or otherwise reported. “Differential diagnosis” includes other medical disorders that may be confused with the subject medical disorder, or other disorders that should be ruled out to confirm a diagnosis of the subject disorder, generally because one or more signs or symptoms are similar between the subject and the differential diagnosis. “Work-Up” includes any type of test performed on a patient, or a patient's fluids, tissues, secretions, or excretions, generally to aid in determining some aspect of the patient's condition or status. “Treatment” includes any medication, surgery, psychotherapy, nursing or other care, dietary or behavioral modification, or any other type of management, recommendation, therapy, or intervention that may be advised by a health care practitioner.

All of these categories need not be included in every embodiment of the invention. Information of practical usefulness or information intended especially as teaching points may be included in any of these categories.

As also illustrated in the examples in FIGS. 1A through 5B, additional categories may be included as appropriate or desired, at any point in the format, such as:
Definition
Epidemiology
Description
Information of particular note (“NB”)
Follow Up
Staging Criteria
Medical Management
Surgical Indications
   Any of these categories may also include information with an object of teaching the user or information of practical usefulness.

The template may also include sections for recording information on a particular patient or making other notes as a user desires, such as:
Patient name, age, sex, and/or other identifying characteristics
Medical record number or other administrative indicator
Vital signs (such as temperature, pulse rate, respiratory rate, and blood pressure)
Laboratory or test data (blood test results, electrocardiograph data, urinalysis results, etc.)
Medications
Notes
   Templates may be further designed especially for use with individual patients by including boxes or other designations by items which a user can mark if, for example, the sign is present or the test is recommended.

Templates relating to a particular medical disorder may be organized into a single device, or a device may include templates specific to various disorders encountered within a particular medical specialty. It might also contain templates that relate to multiple specialties, gathered together in sections and indexed for convenient access by a user. It may be formatted for use with an individual patient or for use as a general reference. It may be formatted simply as a device for recordation of information by a user.

A printed or other type of form of the device, or multiple copies of a form, specific to a particular disorder may be provided, to which a user can add notes and information of special interest to the user or relating to an individual patient. Multiple copies of such forms may be bound together but made so that individual copies can be easily separated without damaging remaining forms.

Forms may be customized by a user for a particular situation and then be changed back to the original presentation for re-use and customization again. This can be accomplished, for example, by coating printed forms with a transparent plastic laminate or by embodiment in computer software.

A template with category headings but no information or data may also be provided. These devices may be implemented as multiple copies of single use forms bound releasably together, as forms capable of repeated erasable uses, or as computer software.

Presentations of information according to the general format of the invention which relate to different disorders likely to be encountered by a practitioner in a particular medical specialty may be collected and bound together. Several of such collections may be bound together, separated by tabs or other indicators for reference and ready location by a user. Templates relating to different medical disorders may be organized alphabetically, by signs or symptoms, by body system, by patient characteristic, or according to some other method, and bound together. As used herein, “bound together” may mean bound together on one edge by conventional book binding, spiral wire binding, ring binding, or glue binding; collection in a computer program; or some other method of permanent, temporary, adjustable, or flexible holding together.

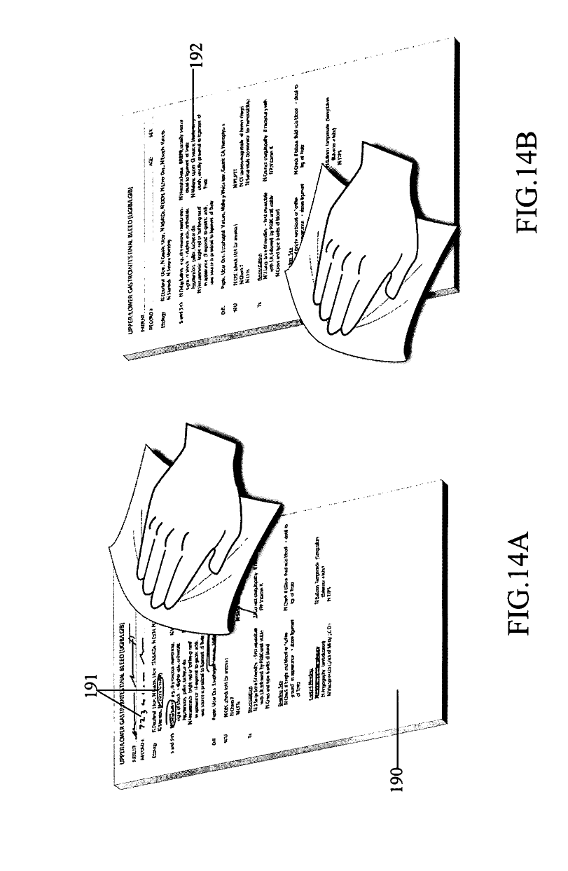

In another embodiment of the invention, shown in FIGS. 14A and 14B, each template for each specific disorder may be printed on paper which may then be encased within transparent plastic. The plastic could be marked by a user during use with a particular patient, then wiped clean, or the marks otherwise removed, for re-use with another patient. FIG. 14A shows a plastic-laminated sheet 190 with marks 191 made by a user. FIG. 14B shows a plastic-laminated sheet 192 after the user's marks have been removed. Such templates may be separate or they may be combined and bound releasably or permanently together by medical specialty or other commonality.

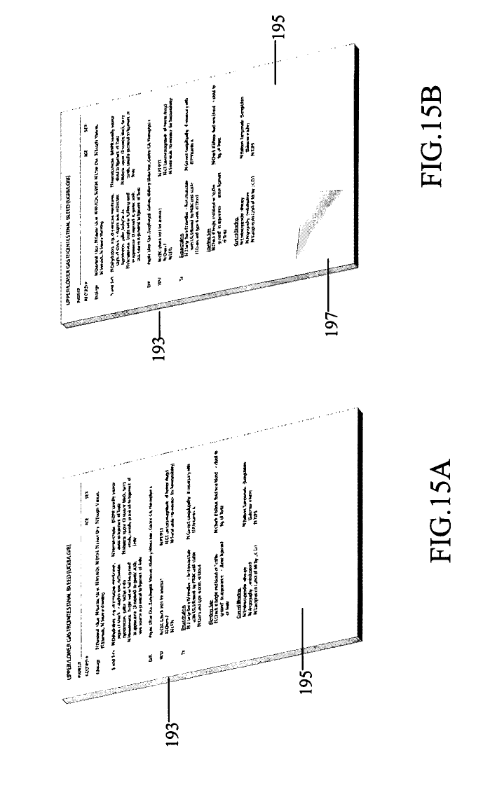

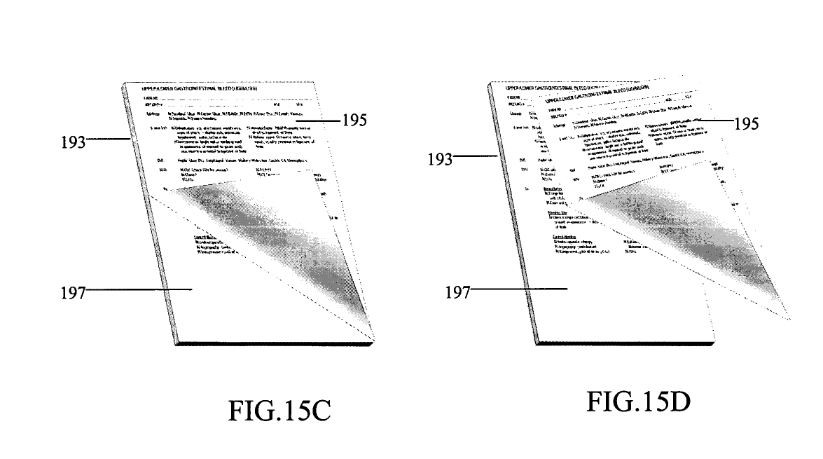

Multiple paper copies of a single template for a specific disorder may be formed into a tablet or pad such that individual copies may be released, used for a particular patient or other purpose, and then put into the patient's record or discarded. FIGS. 15A, 15B, 15C, and 15D illustrate this embodiment. FIG. 15A shows a tablet 193 composed of multiple copies of a printed template with a topmost sheet 195. FIGS. 15B, 15C, and 15D show the topmost sheet 195 being removed from the tablet 193, leaving the tablet 193 with a different sheet 197 on top. A tablet or pad may consist of multiple copies of a single template or, alternatively, multiple copies of multiple templates may be combined into a single tablet or pad, preferably with dividers or some other type of designation between types of templates.

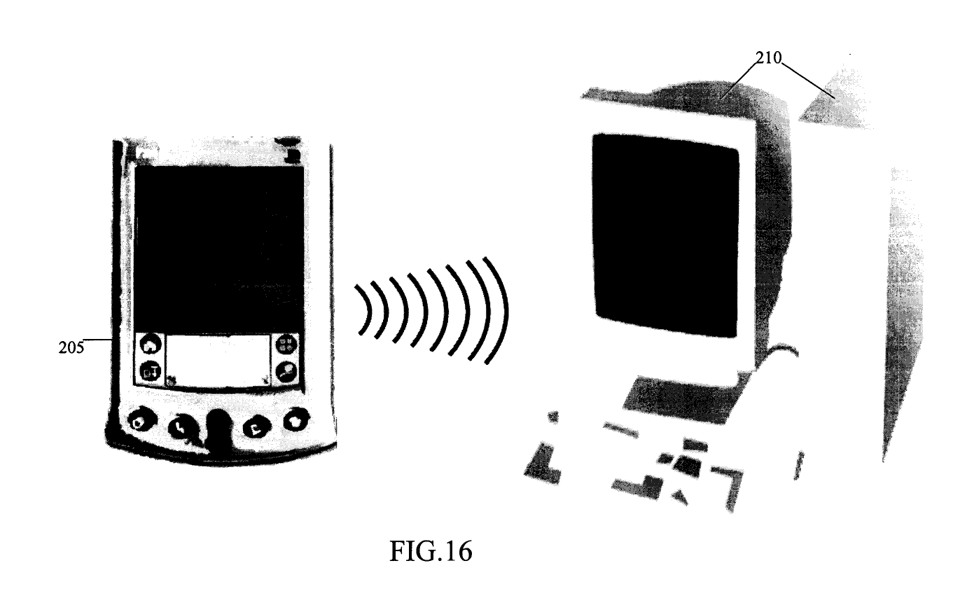

In yet another embodiment of the invention, templates may be coded into software for a computer. Such software may be usable in or accessible from a network system, a personal computer, a hand-held computer, or another electronic device. FIG. 16 illustrates transmission of information according to the invention from a hand-held electronic device 205 to a personal computer 210 connected to a network. One form of this embodiment may enable a user to input information specific to an individual patient onto a template for a medical disorder. If desired, other users may be allowed to access or modify an individualized template, or the original user may access and modify the individualized template from another computer or another location. The template may be formatted to permit electronic transfer from a user to another person, such as another caregiver or a laboratory technician. The computerized template may be printable or may be able to be added to or integrated with a patient's computerized record or other records. The original standard template preferably would remain available in the computer software for repeated uses and customizations and would be accessible for modification as medical knowledge advances or for customization to a health care practice or user.

Thus is presented an invention which can train new health care practitioners, save health care practitioners valuable time, improve medical care, and help prevent mistakes by health care professionals due to inappropriate diagnoses, omitted or improper tests or treatments, or miscommunication.

### EXAMPLES OF SYSTEMS AND METHODS OF USE OF THE INVENTION

The invention may be used starting before a patient is even seen by a health care practitioner. For example, a psychiatrist may be called to consult on a patient with depression. The psychiatrist may have a pad of printed reference devices on depressive disorder according to the invention by his telephone. He may record the patient's name on a copy of the device and mark the device to indicate signs or symptoms the patient is described as exhibiting, use the device to remind him to ask about potential non-psychiatric causes of the depression, and mark the diagnostic tests he would like performed on the patient. The device may remind him to also order initial treatment such as monitoring for suicide. The marked device could be given to a medical resident on the psychiatrist's service team to initiate the patient's psychiatric examination. The resident would not have to decipher the psychiatrist's potentially illegible handwriting, but would be able to clearly see what the psychiatrist wished to communicate. By reviewing the reference device, the resident could learn about the epidemiology of depression and how often suicides occur among depressed persons. The template could explain to the resident theories as to why depression is thought to occur. The resident would be able to understand from the template why certain blood tests could be ordered that might otherwise appear irrelevant to a mental disorder. The resident could also predict how, if depressive disorder is confirmed, the patient is likely to be treated.

The invention may also be used, as another example, by a health care practitioner examining a patient in an emergency room. At the patient's bedside, the practitioner could use a handheld computing device to pull up a template for the medical disorder the practitioner tentatively believes the patient to be experiencing, such as peptic ulcer disease. The template would display information on the etiology of both gastric and duodenal ulcers. The template would trigger the practitioner to ask the patient about use of non-steroidal anti-inflammatory drugs and tobacco, ingestion of alcohol and caffeine, and whether the patient's pain is relieved or worsened by eating. Using the device, the practitioner may immediately be able to consider either a gastric or duodenal ulcer more likely based on the patient's answers and the different usual patient age ranges for each ulcer location that are provided in the template. The practitioner may make entries on the device to indicate the patient's signalment, signs, symptoms, and history, and mark the tests to be conducted to confirm the diagnosis and further evaluate the patient. The practitioner could then transmit the template electronically to the patient's record and other services within the hospital responsible for performing the testing, as well as the patient's attending or primary physician. Information about the patient with an emergency would thus be recorded and communicated quickly, accurately, and efficiently.

Alternate embodiments of the invention which implement the invention as articles, methods, systems, hardware, firmware, software, or a combination thereof, as well as arranging the template and/or the data in a different fashion, will be apparent to those skilled in the art and are also within the scope of the invention.

### SPECIFIC EXAMPLES OF TEMPLATES

Representative examples of medical specialty areas that may be a source for collections and presentations of information according to this invention include: internal medicine, adult psychiatry, child psychiatry, adolescent psychiatry, and neurology. These are examples only; the invention is not intended to be limited to these areas. The invention may comprise collections from other medical specialties, body systems, or commonalities in addition to or instead of these areas.

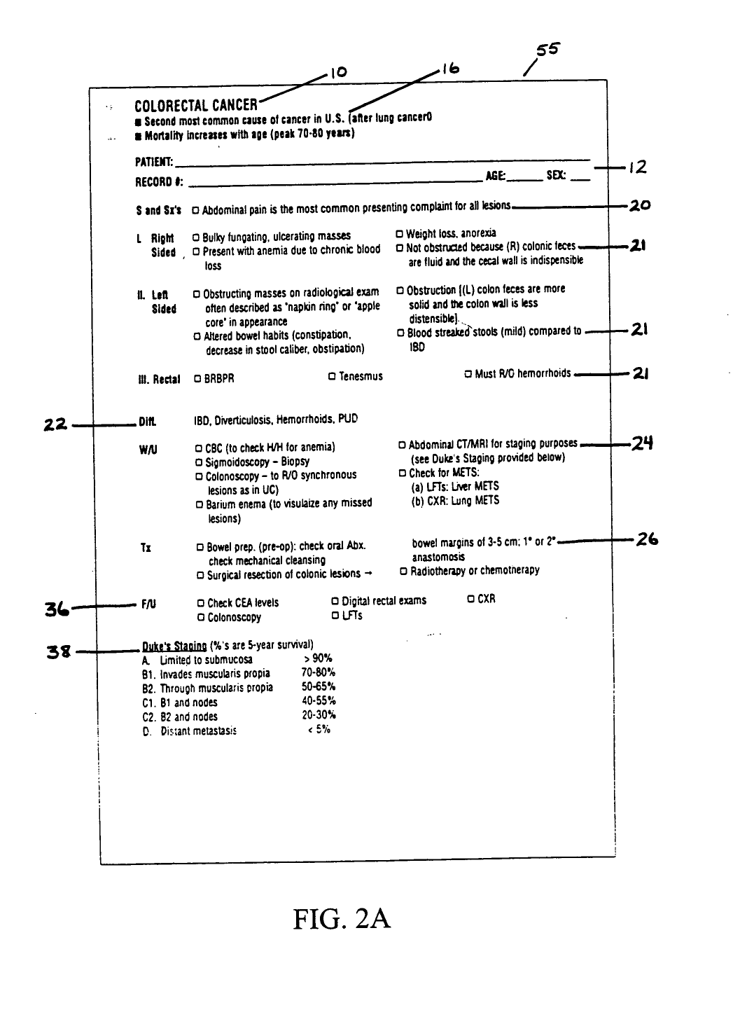

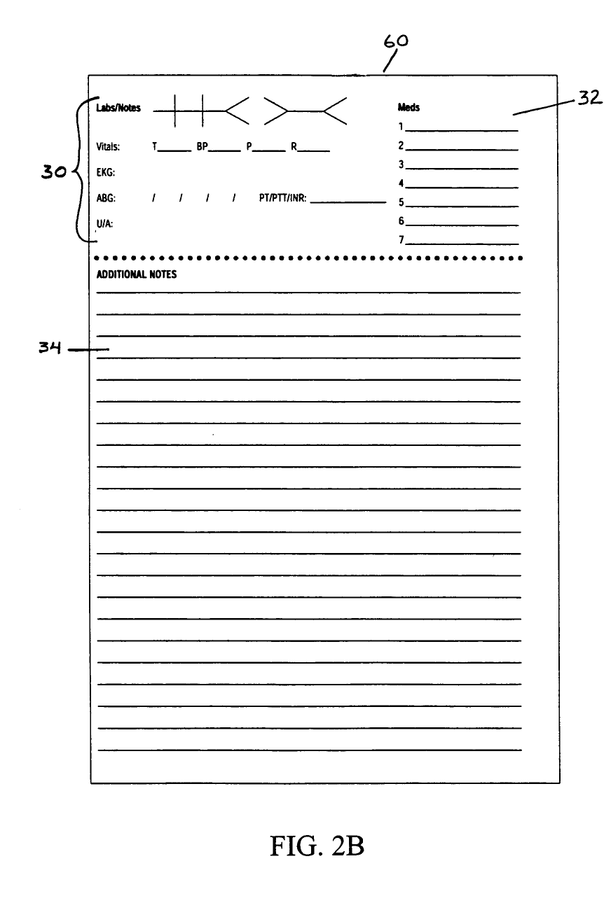

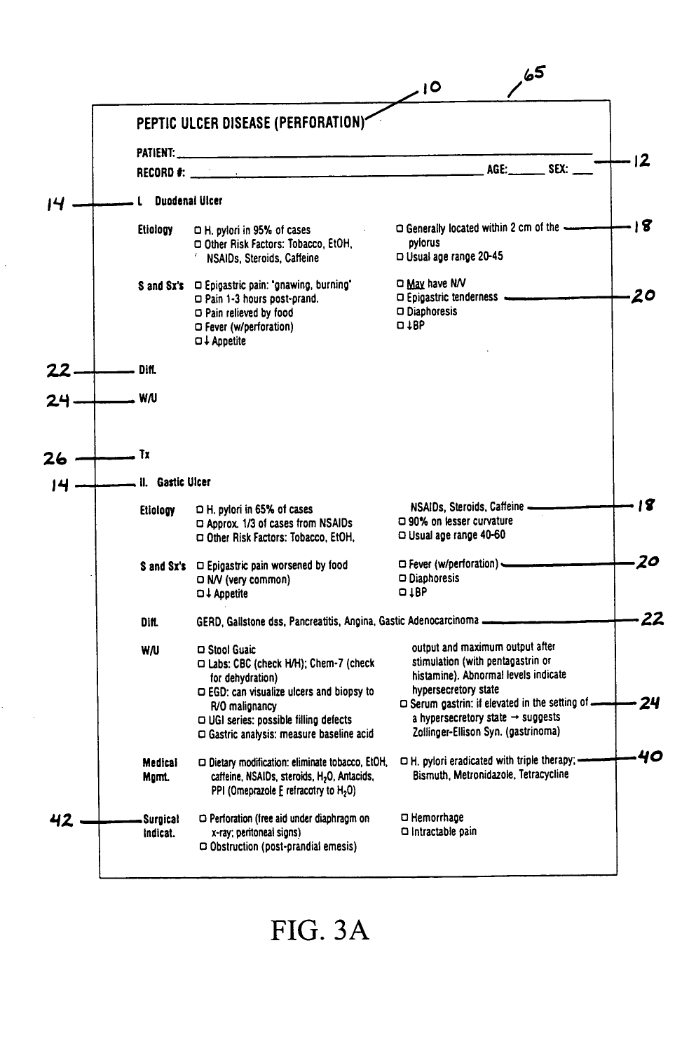

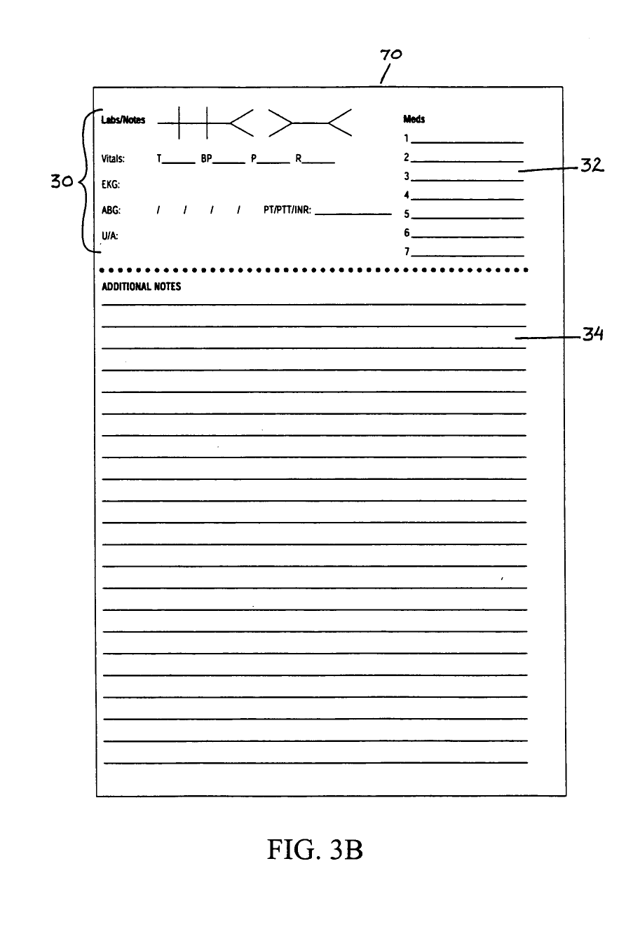

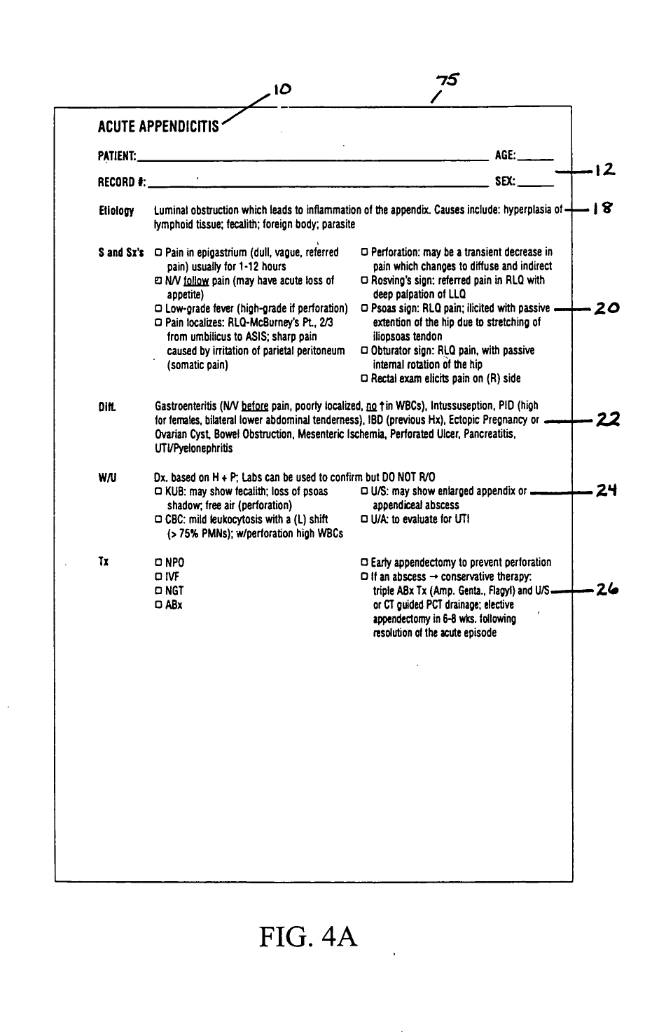

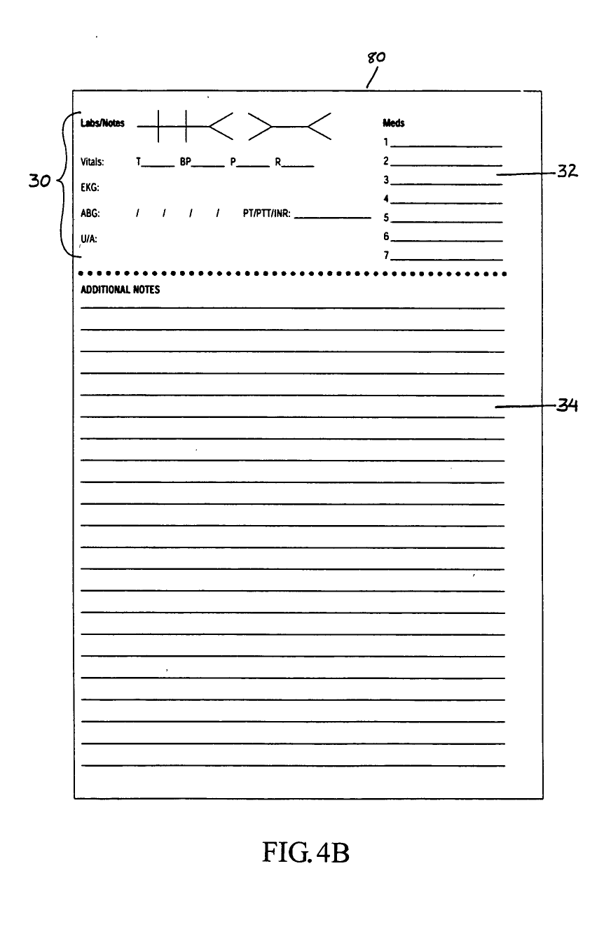

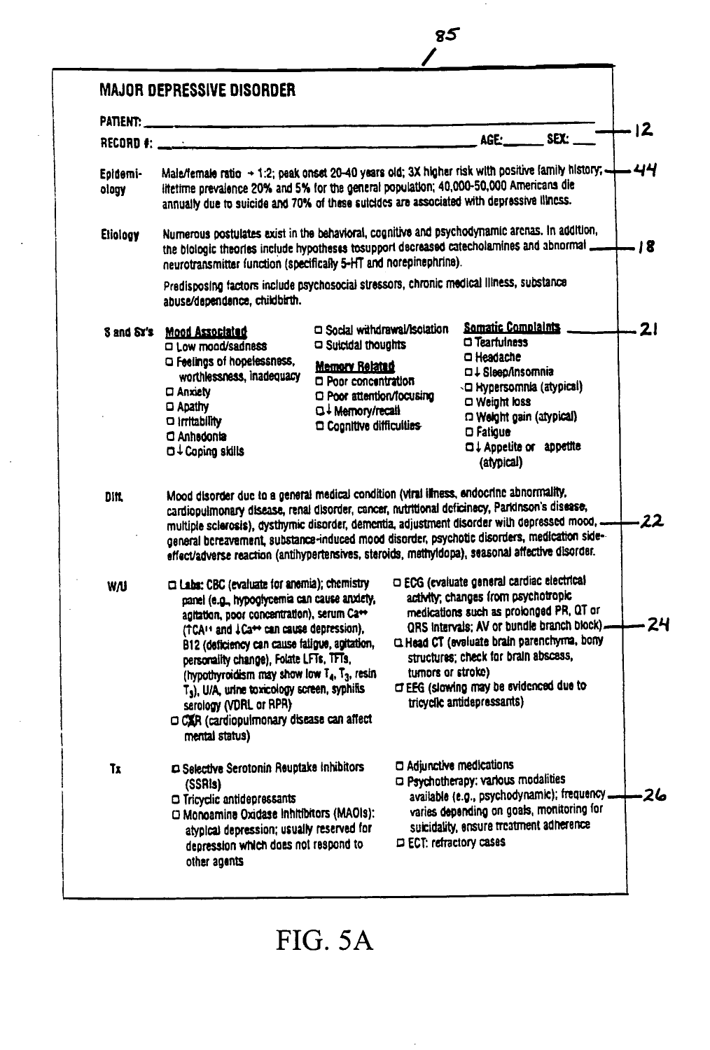

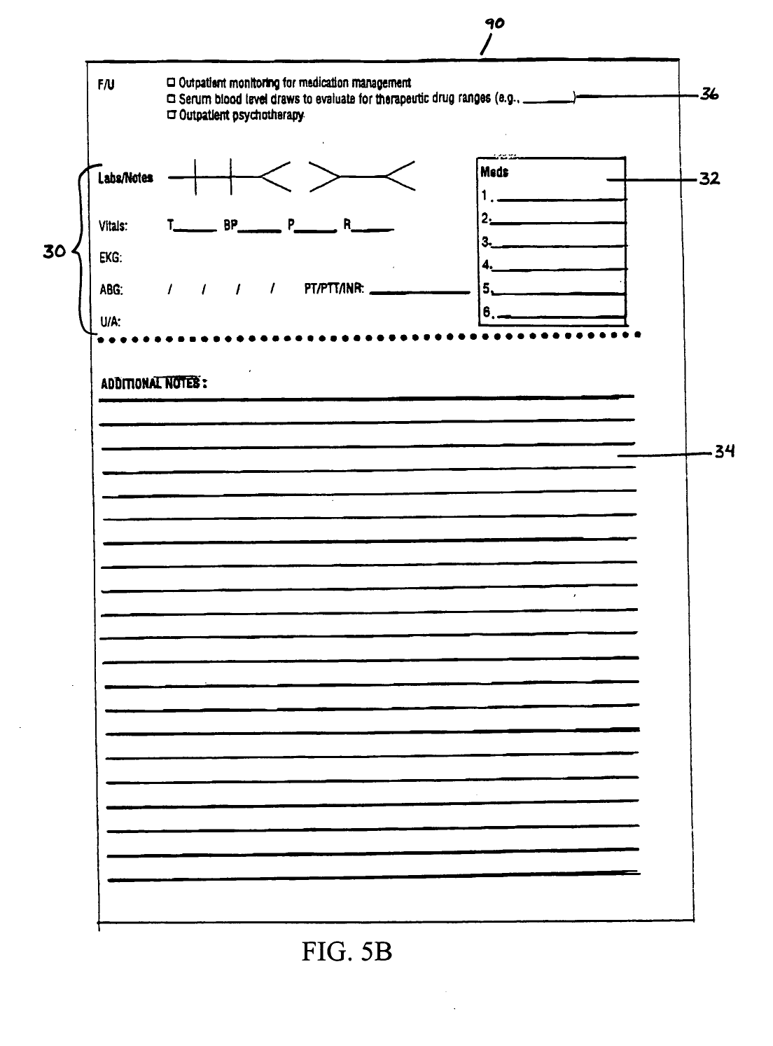

Representative examples of specific pathologies or medical conditions for which information may be collected, organized, and presented according to the invention include: acute appendicitis (see FIGS. 4A and 4B), biliary colic (see FIGS. 1A and 1B), acute cholangitis (see FIG. 1B), acute cholecystitis (see FIGS. 1A and 1B), cholelithiasis (see FIGS. 1A and 1B), colorectal cancer (see FIGS. 2A and 2B), diverticulitis, duodenal ulcer (see FIGS. 3A and 3B), gastric ulcer (see FIGS. 3A and 3B), gastroesophageal reflux disease, diverticulitis, upper gastrointestinal bleed, lower gastrointestinal bleed, inflammatory bowel disease, acute pancreatitis, small bowel obstruction, large bowel obstruction, and major depressive disorder (see FIGS. 5A and 5B). These are examples only; the invention is not intended to be limited to these disorders, nor must the invention include any of these disorders.

A single template may be limited to a specific disorder, as shown in FIGS. 5A and 5B, or disorders with a common characteristic may be included in the same template, organized either consecutively, as shown in FIGS. 1A and 1B, or concurrently, as shown in the signs and symptoms section of FIGS. 2A and 2B. Representative examples of similar disorders which may be included in a single template according to the invention include: small bowel intestinal obstruction and large bowel intestinal obstruction; upper gastrointestinal bleed and lower gastrointestinal bleed; duodenal ulcer and gastric ulcer on a template for peptic ulcer disease; cholelithiasis/biliary colic, acute cholecystitis, and acute cholangitis on a template for gallstone diseases; and right-sided, left-sided, and rectal on a template for colorectal cancer. These are examples only; these or other disorders may be combined in any of a variety of ways consistent with the invention.

FIGS. 1A, 1B, 2A, 2B, 3A, 3B, 4A, 4B, 5A, and 5B illustrate examples of presentations of information for particular medical disorders according to the invention. FIGS. 1A and 1B show first and second parts, respectively, of a form of presentation of information on gallstone diseases, including cholelithiasis, biliary colic, acute cholecystitis, and acute cholangitis. As an illustrative example of an embodiment of the invention, FIG. 1A shows a template in page format 5. The illustration depicts: the title 10 of the medical disorder or group of medical disorders that is the subject of the template; a field 12 for recording data identifying a patient; a title 14 of a specific disorder within the group of medical disorders; descriptive information 16 about the specific medical disorder; information 18 relating to the etiology of the disorder; a list 20 of signs and symptoms of the disorder; differential diagnoses 22 for the disorder; a list 24 of tests and work-up procedures used to confirm or evaluate the disorder; a list 26 of treatments for the disorder; and information 28 of particular note about the disorder. FIG. 1B illustrates another page 50 of the embodiment and fields for recording data about the patient, including laboratory test results and signs 30 determined from a physical examination of the patient; a field 32 for recording medications that the patient is taking or receiving; and a field 34 for recording additional notes, comments, or information relating to the disorder or the patient. FIG. 1B also illustrates separation of signs and symptoms into groups 21 that commonly occur together or are associated with a particular form of the disorder.

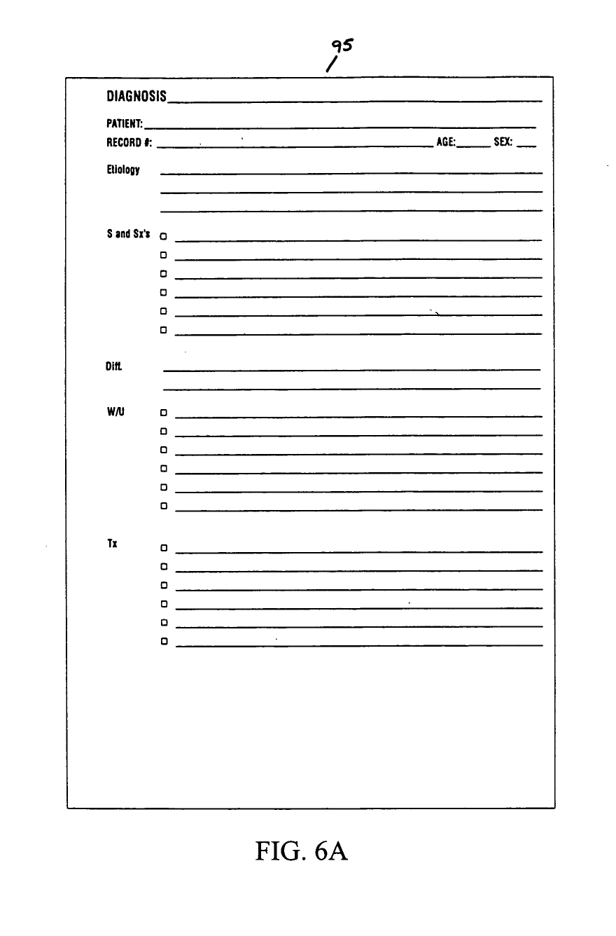

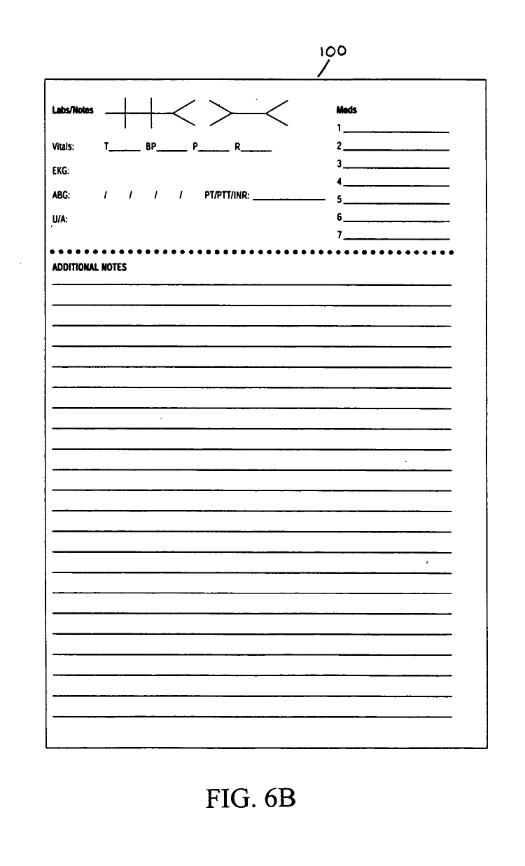

FIGS. 2A and 2B show first 55 and second 60 parts, respectively, of a form of presentation of information on colorectal cancer, including right-sided, left-sided, and rectal. FIGS. 2A and 2B illustrate many of the same features of the invention shown in FIGS. 1A and 1B, and in addition, FIG. 2A illustrates a section 36 for listing follow-up procedures used for patients with the medical disorder and a section 38 describing staging criteria used with the disorder. FIGS. 3A and 3B show first 65 and second 70 parts, respectively, of a form of presentation of information on peptic ulcer disease, including duodenal ulcer and gastric ulcer. FIGS. 3A and 3B show many of the features of the invention also shown in previous figures and additionally FIG. 3A illustrates a section 40 for describing medical management procedures for patients with the disorder and a section 42 for describing indications for performing surgery in patients with the disorder. FIGS. 4A and 4B show first 75 and second 80 parts, respectively, of a form of presentation of information on acute appendicitis. FIGS. 5A and 5B show first 85 and second 90 parts, respectively, of a form of presentation of information on major depressive disorder. FIG. 5A illustrates showing an additional feature of the invention, information 44 on the epidemiology of the disorder. While each of these sets of figures shows a form of template embodied in two parts, the invention is not intended to be limited to two-part presentations. Presentations according to the invention may comprise more or fewer than two parts, or may be electronic and not separated into parts at all. These figures are examples only; presentations according to the invention need not be exactly like these examples even for the same disorders, and may be for any medical or psychiatric disorder. FIGS. 6A and 6B show first 95 and second 100 parts, respectively, of a form of template that may be used by a user of the invention to organize and record information relating to an individual patient or to a specific disorder.

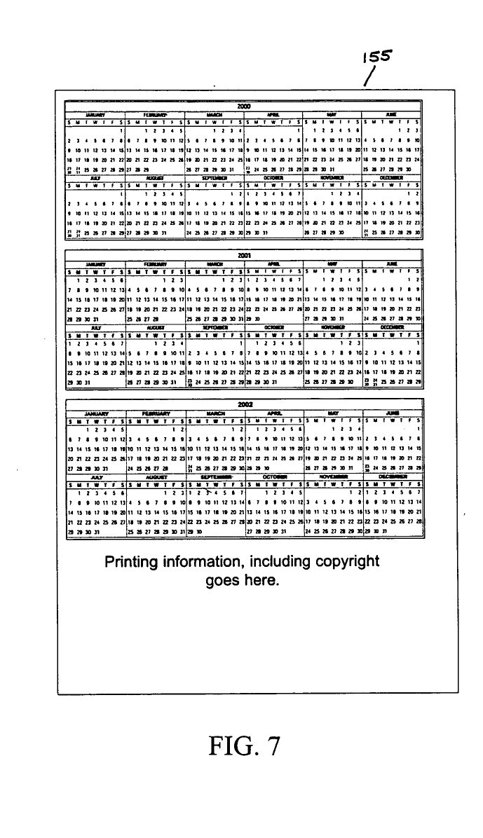

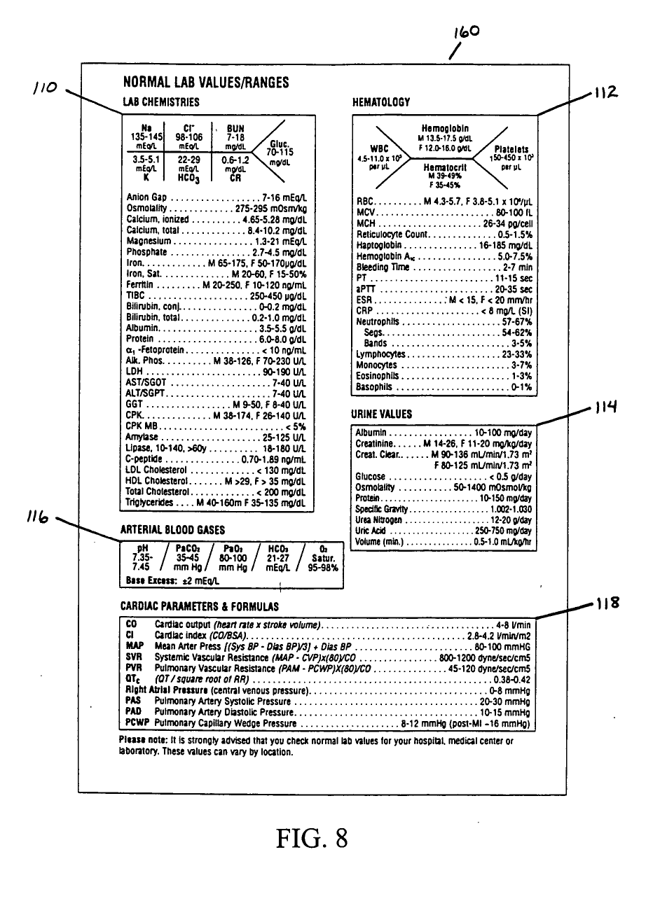

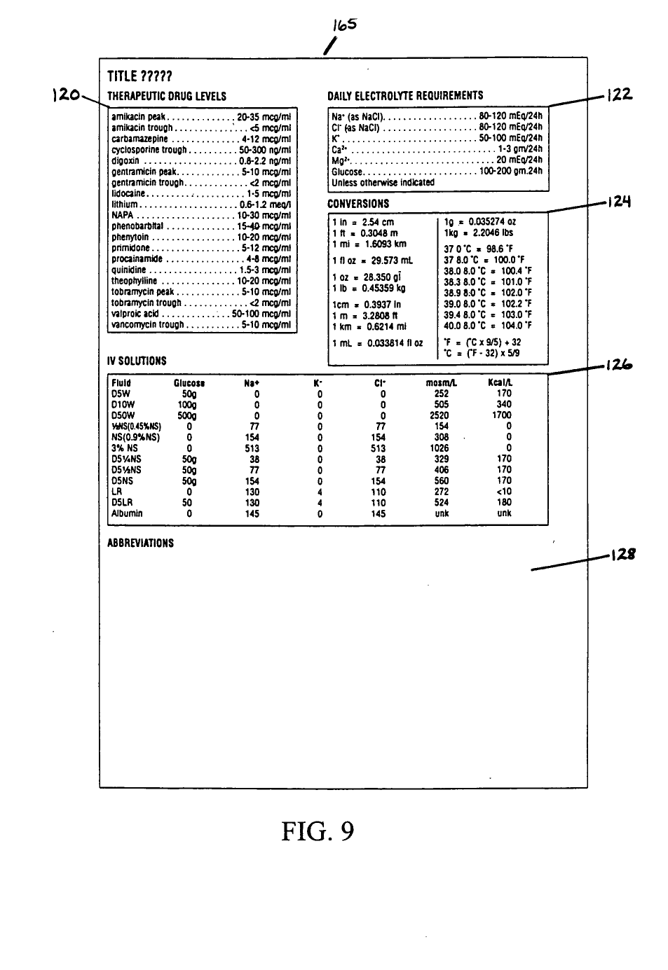

FIGS. 7 through 13 illustrate sample embodiments of presentations of reference information and charts that may be included in the invention. FIG. 7 shows an example of a page 155 of an embodiment of the invention illustrating a calendar displaying three years. FIG. 8 shows a page 160 of an embodiment illustrating the inclusion in the invention of lists of medical laboratory tests and measurements with corresponding values or ranges of results for each test or measurement that are considered normal. The figure illustrates sections 110, 112, 114, and 116 providing information on laboratory chemistry tests on blood serum, hematology tests, tests on urine, and tests on arterial blood, respectively. This figure also shows a section 118 providing reference information on cardiac parameters and formulas. The invention, however, may include any combination or presentation of medical tests or measurements and related values or ranges. Different types of reference information may, but need not be, enclosed within separate boxes as shown to increase ease of use. FIG. 9 illustrates a page 165 of an embodiment of the invention showing inclusion of lists of various types of reference information useful in a medical practice, including a list 120 of the therapeutic levels of commonly administered drugs, a list 122 of the daily requirements for essential electrolytes, a list 126 of the compositions of various common solutions given parenterally, and a list 124 of conversions between metric and English measurements and Centigrade and Fahrenheit temperatures. This figure also illustrates a means 128 for providing a user with a place to record additional information, such as abbreviations. Any data useful to a medical practitioner may be included.

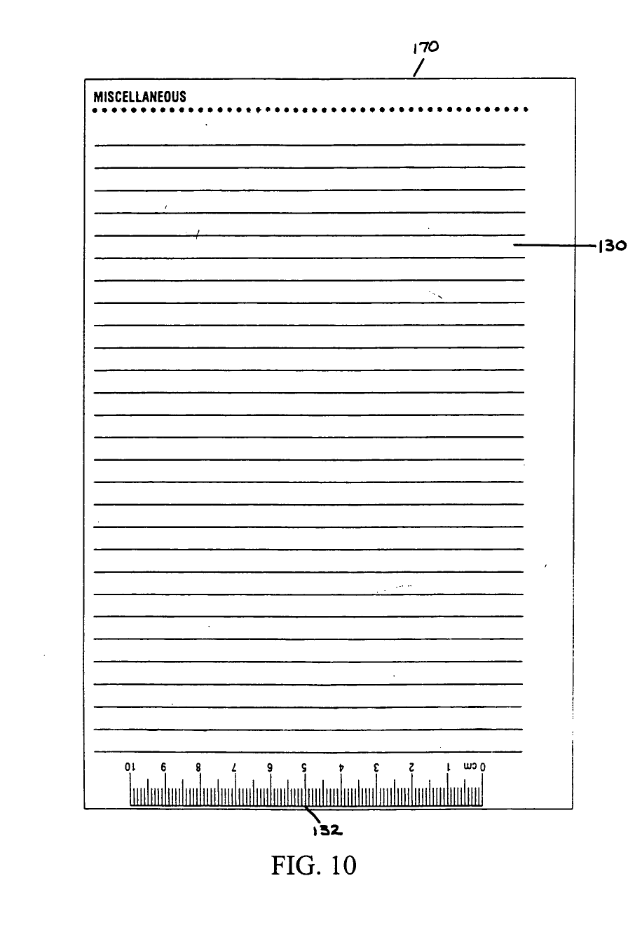

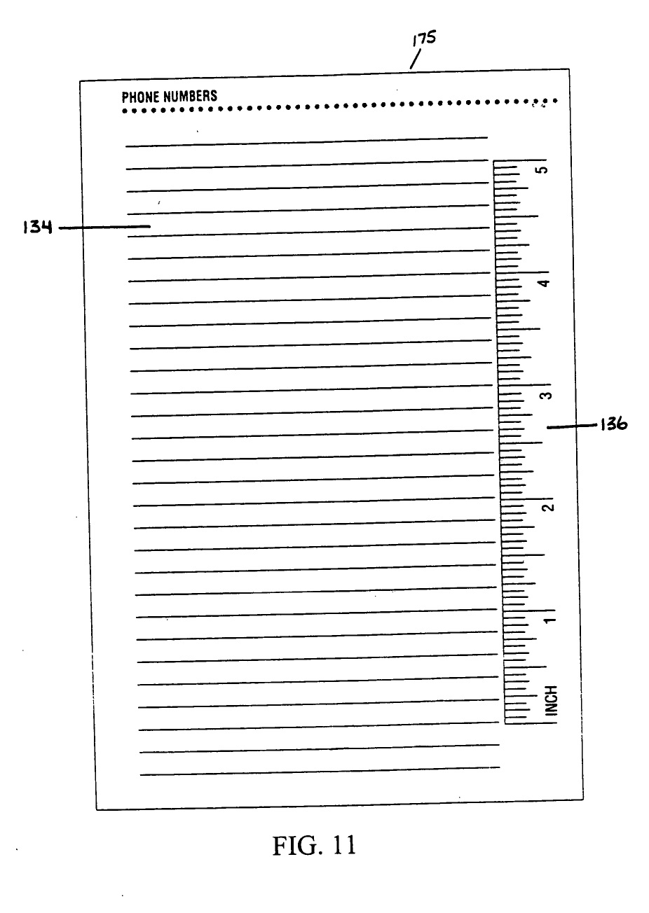

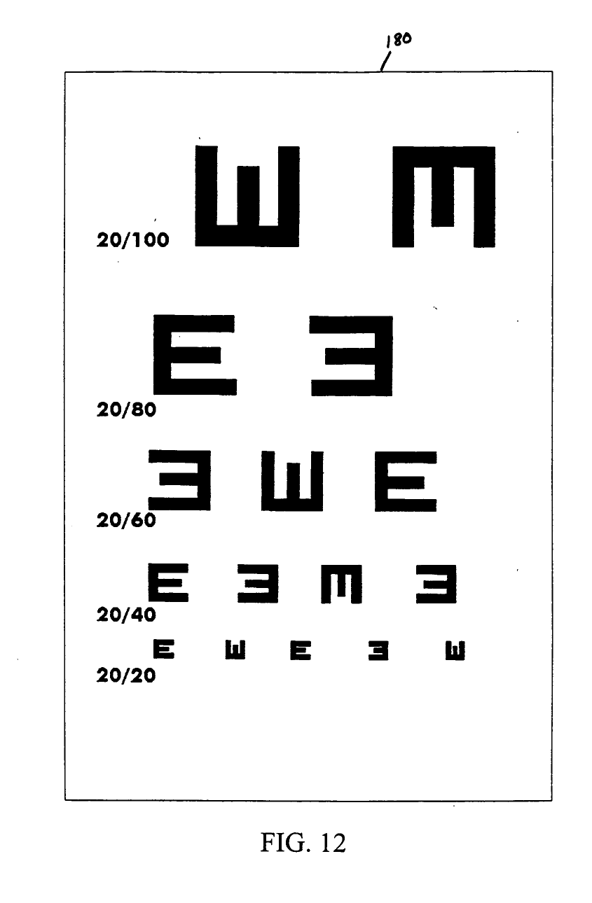

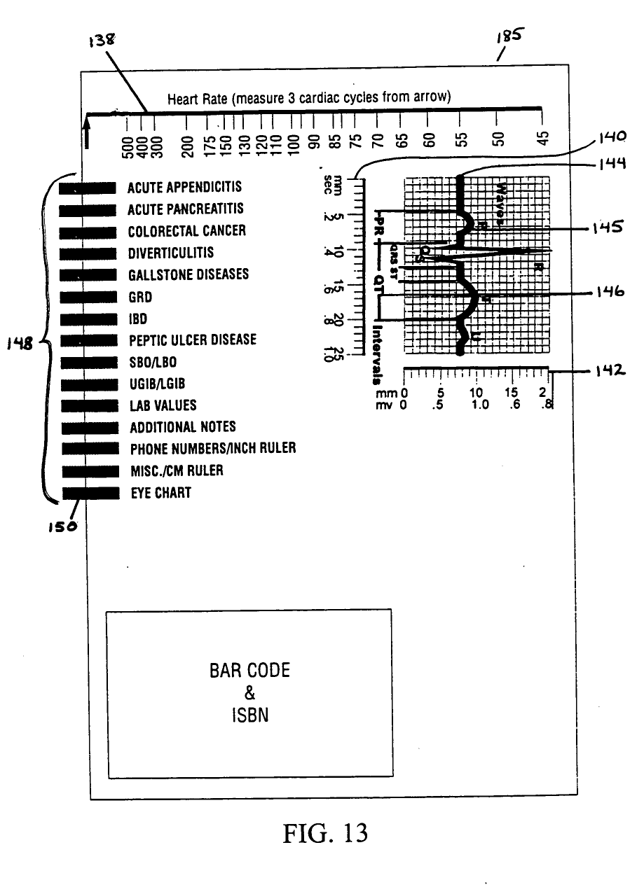

FIG. 10 shows a form 170 having a field 130 that may be used by a user of the invention for recording notes. This figure also illustrates an example of a metric ruler 132 and how it may be included in the invention to increase the practical usefulness of the invention. FIG. 11 shows a page 175 of an embodiment of the invention with an example of an English system ruler 136 and how it may similarly be included in the invention. This figure also shows a sample of a form 134 that may be used by a user of the invention for recording information of particular usefulness to that user such as telephone numbers. Reference information may be combined and presented in any format; the examples shown in these figures are not intended to limit the invention. FIG. 12 depicts a form of chart 180 used to test visual acuity which may be included in the invention; any type of similar vision chart may be included in the invention if desired. FIG. 13 illustrates a form of page 185 that may be incorporated into the invention which includes examples of rulers for measuring electrocardiographic data, including a ruler 138 for measuring heart rate, a ruler 140 for measuring segment intervals, and a ruler 142 for measuring wave amplitudes, and shows how such rulers can be included in the invention. It also shows a model electrocardiographic trace of a heartbeat 144, with each component wave 144 and interval 146 labeled, as another example of useful reference information that may be included in the invention. This figure also shows a way 148 in which the contents of an embodiment of the invention may be depicted to enable a user to quickly access the part of the invention desired. The index 148 shown may feature a band 150 of a different color for each template or reference chart and may correspond to templates or charts with pages or page edges of the same colors elsewhere in the reference.

The invention may comprise templates that are blank, with no information but only category captions and space, and perhaps lines, on which a user can record information relating to a medical disorder or an individual patient. An example of such a template is shown in FIGS. 6A and 6B.

Included in a template or elsewhere in the handbook may be useful reference data, such as normal values or value ranges for laboratory tests; therapeutic or toxic drug levels; normal requirements for electrolytes, nutrients, fluids, or other similar necessities; English/Metric, Fahrenheit/Centigrade, or other conversion tables; compositions of different types of fluid solutions; rulers for measuring length or electrocardiographs; vision charts; calendars; or other useful data or information. Space may also be provided to permit a user to insert notes or data of use or interest to the user, such as telephone numbers or miscellaneous information, as shown in FIGS. 10 and 11.

Bits of information that may be entries in templates according to the invention may be pertinent for diagnosis, treatment, or training purposes. The information may be of practical use to a health care practitioner or purely academic.

Common (or defined) abbreviations for categories, tests, or terms may be used instead of complete words or phrases in any part of the invention, and are encouraged if readily understandable by the intended user or defined within the embodiment of the invention. Representative examples of such abbreviations which may be used include: “ABD” to mean abdomen or abdominal; “ABG” to mean arterial blood gases; “ABx” to mean antibiotics; “BP” to mean blood pressure; “CBC” to mean complete blood count; “CBD” to mean common bile duct; “CXR” to mean chest x-ray (thoracic radiograph); “Diff.” to mean differential diagnoses; “Dx” to mean diagnosis; “EEG” to mean electroencephalograph; “EKG” or “ECG” to mean electrocardiograph; “EtOH” to mean ethanol (alcohol); “F/U” to mean follow up; “GB” to mean gall bladder; “H/H” to mean hematocrit and hemoglobin; “Hx” to mean history; “IBD” to mean inflammatory bowel disease; “IVF” to mean intravenous fluids; “(L)” to mean left; “LLQ” to mean left lower quadrant; “NB” to mean note well; “NPO” to mean nil per os (nothing by mouth); “NSAID” to mean non-steroidal anti-inflammatory drug; “NN” to mean nausea/vomiting; “(R)” to mean right; “R/O” to mean rule out; “RUQ” to mean right upper quadrant; “S and Sx's” to mean signs and symptoms; “T” to mean temperature; “TPN” to mean total parenteral nutrition; “Tx” to mean treatment; “U/A” to mean urinalysis; “UGI” to mean upper gastrointestinal; “U/S” to mean ultrasound; “WBC” to mean white blood cells; “W/U” to mean work up. Many other abbreviations may also be used or may be used instead of these examples and will be evident to persons with medical education or experience. Arrows may be used to indicate progression, a cause and effect relationship, increase or decrease, high or low, or other meaning evident from the direction of the arrow and the context in which it is used.

The figures show examples of bits of information that may be entries in particular template categories on forms or reference devices for medical disorders which may be included in an embodiment of the invention.

Examples of information that may be included in a “Definition or Epidemiology” category are: (a) on a template for Acute Cholecystitis, as shown in FIG. 1A: “Acute inflammation of the GB caused by a protracted stone in the cystic duct; can cause sepsis, GB necrosis or abscess”; (b) on a template for Major Depressive Disorder, as shown in FIG. 5A: “Male/female ratio 1:2; peak onset 20-40 years old; 3× higher risk with positive family history; lifetime prevalence 20% and 5% for the general population; 40-50,000 Americans die from suicide annually and 70% of these suicides are associated with depressive illness.”

Examples of information that may be included in an “Etiology” category are: (a) on a template for Duodenal Ulcer, as shown in FIG. 3A: “H. pylori in 95% of cases; other risk factors: tobacco, EtOH, NSAIDs, steroids, caffeine; generally located within 2 cm of the pylorus; usual age range 20-45”; (b) on a template for Acute Appendicitis, as shown in FIG. 4A: “Luminal obstruction which leads to inflammation of the appendix. Causes include: hyperplasia of lymphoid tissue; fecalith; foreign body; parasite.”

Examples of information that may be included in a “Signs and Symptoms” category are: (a) on a template for Left Sided Colorectal Cancer, as shown in FIG. 2A: “(i) Obstructing masses on radiological exam often described as ‘napkin ring’ or ‘apple core’ in appearance, (ii) Altered bowel habits (constipation, decrease in stool caliber, obstipation), (iii) Obstruction because (L) colon feces are more solid and the colon wall is less distensible, (iv) Blood streaked stools (mild) compared to IBD”; (b) on a template for Acute Appendicitis, as shown in FIG. 4A: “(i) Pain in epigastrium (dull, vague, referred pain) usually for 1-12 hours, (ii) N/V following pain (may have acute loss of appetite), (iii) Low-grade fever (high-grade if perforation), (iv) Pain localizes: RLQ-McBurney's Point, 2/3 from umbilicus to ASIS; sharp pain caused by irritation of parietal peritoneum (somatic pain), (v) Perforation: may be a transient decrease in pain which changes to diffuse and indirect, (vi) Rosving's sign: referred pain in RLQ with deep palpation of LLQ, (vii) Psoas sign: RLQ pain; elicited with passive extension of the hip due to stretching of iliopsoas tendon, (viii) Obturator sign: RLQ pain, with passive internal rotation of the hip, (ix) Rectal exam elicits pain on (R) side.”

Examples of information that may be included in a “Differential Diagnoses” category are: (a) on a template for Acute Cholecystitis, as shown in FIG. 1A: “Biliary colic, cholangitis, GERD, MI, acute appendicitis, peptic ulcer, pneumonia”; (b) on a template for Major Depressive Disorder, as shown in FIG. 5A: “Mood disorder due to a general medical condition (viral illness, endocrine abnormality, cardiopulmonary disease, renal disorder, cancer, nutritional deficiency, Parkinson's disease, multiple sclerosis), dysthymic disorder, dementia, adjustment disorder with depressed mood, general bereavement, substance-induced mood disorder, psychotic disorders, medication side-effect/adverse reaction (antihypertensives, steroids, methyldopa), seasonal affective disorder.”

Examples of information that may be included in a “Laboratory and Other Work-Up” category are: (a) on a template for Cholelithiasis and Biliary Colic, as shown in FIG. 1A: “ABD U/S: may show gallstones; EKG: R/O MI; CXR; UGI series to R/O hiatal hernia or ulcer”; (b) on a template for Colorectal Cancer, as shown in FIG. 2A: “CBC (to check H/H for anemia); sigmoidoscopy/biopsy; colonoscopy: R/O synchronous lesions as in UC); barium enema (to visualize any missed lesions); abdominal CT/MRI for staging purposes; check for METS: (a) LFTs: liver METS, (b) CXR: lung METS.”

Examples of information that may be included in a “Treatment” category are: (a) on a template for Acute Cholecystitis, as shown in FIG. 1A: “NPO, IVF, IV ABx (e.g., Mefoxin), pain management”; (b) on a template for Acute Appendicitis, as shown in FIG. 4A: “NPO; IVF; NGT; ABx; early appendectomy to prevent perforation; if an abscess→conservative therapy: triple ABx Tx (amp., genta., Flagyl) and U/S or CT guided PCT drainage; elective appendectomy in 6-8 wks. following resolution of the acute episode.”

FIG. 3A shows an example of use of a “Medical Management” category: “Dietary modification: eliminate tobacco, EtOH, caffeine, NSAIDs, steroids, H₂O, antacids; H. pylori eradicated with triple therapy: bismuth, metronidazole, tetracycline.” FIG. 3A also shows an example of use of a “Surgical Indications” category: “Perforation (free air under diaphragm on x-ray, peritoneal signs); obstruction (post-prandial emesis); hemorrhage; intractable pain.”

FIG. 5B shows an example of use of a “Follow Up” category: “Outpatient monitoring for medication management; serum blood level draws to evaluate for therapeutic drug ranges; outpatient psychotherapy.”

FIG. 1A shows an example of use of a “NB” category: “Most stones are cholesterol stones (75%) and radiolucent; pigmented stones due to hemolysis (e.g., hyperbilirubinemia) are radiopaque.”

It is to be understood that the above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

1. A method for facilitating the evaluation, diagnosis and treatment of a patient suspected of having a medical disorder, comprising:
determining attributes of the patient and making a preliminary diagnosis based on the attributes that a patient has a particular medical disorder;
selecting a source of information about the particular medical disorder;
comparing the attributes of the patient to the information on the selected source of information;
indicating a portion of the information from the selected source that matches or does not match an attribute of the patient;
recording data identifying the patient; and
simultaneously displaying the selected information, matching indicia and patient data.

2. The method of claim 1, wherein the information in the source about a medical disorder comprises at least one sign or symptom of the medical disorder, at least one test used to confirm or evaluate the medical disorder, and at least one treatment for the medical disorder.

3. The method of claim 1, wherein said source comprises a template containing information relating to one or more medical disorders and having a field for the entry of patient information.

4. The method of claim 3, wherein the template is a printed sheet.

5. The method of claim 4, wherein the sheet is erasable and reusable.

6. The method of claim 3, wherein the source is a computerized database and the template is a computer-generated visual display.

7. The method of claim 1, further comprising transmitting the selected information, matching indicia and patient data from a health care practitioner making the selection, indication or recordation to another health care practitioner or to the patient's medical record.

8. A method for managing patient medical information using a computer system having data storage means and a graphical user interface including a display, data input means, and a selection device, the method comprising:
retaining in the storage means a database of information related to medical conditions;
using the selection device to retrieve from the database information relating to a medical condition;
using the data input means to enter patient data into the storage means; and
simultaneously displaying the retrieved information and the patient data on the display.

9. The method of claim 8, further comprising displaying additional reference information not limited to the medical condition or patient data.

10. The method of claim 8, wherein the database comprises information on the etiologies of said medical conditions.

11. The method of claim 8, wherein the database comprises information on the signs or symptoms of the medical conditions.

12. The method of claim 11, wherein the displayed patient data and the retrieved medical condition information associates one of said signs or symptoms with the patient.

13. The method of claim 8, wherein the database comprises information on differential diagnoses of the medical conditions.

14. The method of claim 8, wherein the database comprises information on diagnostic tests or work-up procedures used to confirm or evaluate the medical conditions.

15. The method of claim 14, wherein the displayed patient data and the retrieved medical condition information associates one of said tests or a portion of said work-up with the patient.

16. The method of claim 8, wherein the database comprises information on the treatment or management of patients with the medical conditions.

17. The method of claim 16, wherein the displayed patient data and the retrieved medical condition information associates one of said treatments or management procedures with the patient.

18. The method of claim 8, wherein said patient data identifies the said patient.

19. A computer-readable medium having computer-executable instructions for facilitating the performance of the method of claim 8.

20. The method of claim 8, wherein the medical information includes at least one type of information selected from each of the following four groups:
(a) definition, epidemiology, etiology, background, or description;
(b) sign, symptom, or abnormality;
(c) differential diagnosis, diagnostic work-up, laboratory test or data; and
(d) treatment, management, follow-up, prognosis, staging criteria, or surgical indication.

21. The method of claim 20, wherein said displayed medical information and patient data associates one of said signs or symptoms with the patient.

22. A device for use in connection with providing health care to a patient, comprising:
a source of information on signs or symptoms associated with a medical disorder, wherein a user can select a sign or symptom relevant to the patient and the device maintains a record of the selection;
a source of information on tests used to determine the presence or severity of the medical disorder, wherein a user can select one of said tests relevant to the patient and the device maintains a record of the selection;
a source of information on treatments for the medical disorder, wherein a user can select a treatment relevant to the patient and the device maintains a record of the selection; and
at least one field associated with said sources of information for entry of patient data.

23. The device of claim 22, wherein the device is a printed template.

24. The device of claim 22, further comprising multiple copies of the templates assembled together such that one copy at a time can be removed by a user without damaging the remaining copies.

25. The device of claim 22, wherein the template is adapted to be marked by a user and the marks can be later removed and the template returned to its original condition and marked again.

26. The device of claim 22, wherein the device is an electronic storage and computing device.

27. The device of claim 26, wherein the device is portable.
